(19) United States
(12) Patent Application Publication
(10) Pub. No.: US 2015/0004103 A1
(43) Pub. Date: Jan. 1, 2015

(54) Tumorspecific PET/MR(T1), PET/MR(T2) and PET/CT contrast agent

(71) Applicants: János BORBÉLY, Debrecen (HU); István HAJDU, Tiszacsege (HU); Magdolna BODNÁR, Debrecen (HU); Zsuzsanna CSIKÕS, Nyirbátor (HU)

(72) Inventors: János BORBÉLY, Debrecen (HU); István HAJDU, Tiszacsege (HU); Magdolna BODNÁR, Debrecen (HU); Zsuzsanna CSIKÕS, Nyirbátor (HU)

(73) Assignee: BBS NANOTECHNOLOGY LTD., Debrecen (HU)

(21) Appl. No.: 14/133,766

(22) Filed: Dec. 19, 2013

Related U.S. Application Data
(60) Provisional application No. 61/840,482, filed on Jun. 28, 2013.

(51) Int. Cl. A61K 49/06; A61K 49/04

(52) U.S. Cl. CPC A61K 49/06; A61K 49/04
USPC 424/9.322; 424/9.42

(57) ABSTRACT

New types of nanoparticle-based dual-modality positron emission tomography/magnetic resonance imaging (PET/MRI) and positron emission tomography/computed tomography (PET/CT) tumorspecific contrast agents have been developed. The base of the new type contrast agents is biopolymer-based nanoparticle with PET, MRI and CT active ligands. The nanoparticle contains at least one polyanion and polycation, which form nanoparticles via ion-ion interaction. The self-assembled polyelectrolytes can transport gold nanoparticles as CT contrast agents, or SPION or Gd(III) ions as MRI active ligands, and are labeled using a complexing agent with gallium as PET radiopharmacon. Furthermore, these dual modality PET/MRI and PET/CT contrast agents are labeled with targeting moieties to realize the tumorspecificity.

This application claims priority to U.S. provisional application Ser. No. 61/840,482, filed Jun. 28, 2013, the entire disclosure of which is hereby incorporated by reference herein.

### FIELD OF THE INVENTION

New types of nanoparticle-based dual-modality positron emission tomography/magnetic resonance imaging (PET/MRI) and positron emission tomography/computed tomography (PET/CT) tumorspecific contrast agents have been developed. The base of the new type contrast agents is biopolymer-based nanoparticle with PET, MRI and CT active ligands. The nanoparticle contains at least one polyanion and polycation, which form nanoparticles via ion-ion interaction. The self-assembled polyelectrolytes can transport gold nanoparticles as CT contrast agents, or SPION or Gd(III) ions as MRI active ligands, and are labeled using a complexing agent with gallium as PET radiopharmacon. Furthermore, these dual modality PET/MRI and PET/CT contrast agents are labeled with targeting moieties to realize the tumorspecificity.

### BACKGROUND OF THE INVENTION

Molecular imaging plays a very important role in molecular or personalized medicine. Molecular imaging enables visualization of the biological targets and understanding its complexities for diagnosis and treatment of the disease. An accurate and realtime imaging of biological targets provides a thorough understanding of the fundamental biological processes and helps to diagnose various diseases successfully. It is difficult to obtain all the necessary information about the biological structure and function of an organ by any single imaging modality among all the existing imaging techniques. Therefore attempts are being made to fuse the advantages of different imaging techniques by combining two or more imaging modalities while reducing their disadvantages.

In the past decade, a wide variety of nanoparticles has been used for diagnostic applications. Use of nanotechnology in diagnostic is very useful because only a small volume of sample is enough to achieve the appropriate low limit of detection. Often the use of nanoparticles in diagnosis is more sensitive than use biomolecules.

Some publications attest to the variety of nanoparticles used in diagnostic. Nanocarriers including magnetic resonance imaging (MRI), computed tomography (CT), single photon emission computed tomography, positron emission tomography, or multifunctional nanoparticles such as PET/MR and SPECT/CT have been disclosed.

The fusion of PET and MRI or PET and CT in a single contrast agent has proved to be beneficial as it gives images of high sensitivity and high resolution and nanoparticles are the ideal devices that allow the integration of several different imaging modalities onto a single platform.

### REFERENCES CITED

Hongmin Chen, Zipeng Zhen, Trever Todd, Paul K. Chu, Jin Xie: Nanoparticles for improving cancer diagnosis. Materials Science and Engineering R 74 (2013) 35-69.
Jin Xie, Seulki Lee, Xiaoyuan Chen: Nanoparticle-based theranostic agents. Advanced Drug Delivery Reviews 62 (2010) 1064-1079.
H. Shokrollahi: Contrast agents for MRI. Materials Science and Engineering C 33 (2013) 4485-4497.
Jain K K: Nanotechnology in clinical laboratory diagnostics. Clin Chim Acta 358 (2005) 37-54.
Hong-Wu Zhang, Li-Qin Wang, Qing-Feng Xiang, Qian Zhong, Lu-Ming Chen, Cai-Xia Xu, Xian-Hong Xiang, Bo Xu, Fei Meng, Yi-Qian Wan, David Y. B: Deng Specific lipase-responsive polymer-coated gadolinium nanoparticles for MR imaging of early acute pancreatitis. Biomaterials 35 (2014) 356-367.
Raoul Kopelman, Yong-Eun Lee Koo, Martin Philbert, Bradford A. Moffat, G. Ramachandra Reddy, Patrick McConville, Daniel E. Hall, Thomas L. Chenevert, Mahaveer Swaroop Bhoj ani, SarahM. Buck, Alnawaz Rehemtulla, Brian D. Ross: Multifunctional nanoparticle platforms for in vivo MRI enhancement and photodynamic therapy of a rat brain cancer. Journal of Magnetism and Magnetic Materials 293 (2005) 404-410.
Don B. Elrod, Ranga Partha, Delia Danila, S. Ward Casscells, Jodie L. Conyers: An iodinated liposomal computed tomographic contrast agent prepared from a diiodophosphatidylcholine lipid. Nanomedicine: Nanotechnology, Biology, and Medicine 5 (2009) 42-45.
Rui Zhang, Chiyi Xiong, Miao Huang, Min Zhou, Qian Huang, Xiaoxia Wen, Dong Liang, Chun Li: Peptide-conjugated polymeric micellar nanoparticles for Dual SPECT and optical imaging of EphB4 receptors in prostate cancer xenografts. Biomaterials 32 (2011) 5872-5879.
Eric A. Simone, Blaine J. Zern, Ann-Marie Chacko, John L. Mikitsh, Eric R. Blankemeyer, Silvia Muro, Radu V. Stan, Vladimir R Muzykantov: Endothelial targeting of polymeric nanoparticles stably labeled with the PET imaging radioisotope iodine-124. Biomaterials 33 (2012) 5406-5413.
Zohreh Varasteh, Irina Velikyan, Gunnar Lindeberg, Jens Sorensen, Mats Larhed, Mattias Sandström, Ram Kumar Selvaraju, Jennie Malmberg, Vladimir Tolmachev, and Anna Orlova: Synthesis and Characterization of a High-Affinity NOTA-Conjugated Bombesin Antagonist for GRPR-Targeted Tumor Imaging. Bioconjugate Chem. 24 (2013) 1144-1153.
Sung-min Kim, Min Kyung Chae, Min Su Yim, Il Ha Jeong, Janggeun Cho, Chulhyun Lee, Eun Kyoung Ryu: Hybrid PET/MR imaging of tumors using an oleanolic acid-conjugated nanoparticle. Biomaterials 34 (2013) 8114-8121.
Jin Xie, Kai Chen, Jing Huang, Seulki Lee, Jinhua Wang, Jinhao Gao, Xingguo Li, Xiaoyuan Chen: PET/NIRF/MRI triple functional iron oxide nanoparticles. Biomaterials 31 (2010) 3016-3022.
Hao-Wen Kao, Yi-Yu Lin, Chao-Cheng Chen, Kwan-Hwa Chi, Der-Chi Tien, Chien-Chung Hsia, Ming-Hsien Lin, Hsin-Ell Wang: Evaluation of EGFR-targeted radioimmuno-gold-nanoparticles as a theranostic agent in a tumor animal model. Bioorganic & Medicinal Chemistry Letters 23 (2013) 3180-3185.

### THE STATE OF THE ART

U.S. Pat. No. 7,976,825 relates to macromolecular contrast agents for magnetic resonance imaging. Biomolecules and their modified derivatives form stable complexes with paramagnetic ions thus increasing the molecular relaxivity of carriers. The synthesis of biomolecular based nanodevices for targeted delivery of MRI contrast agents is described. Nanoparticles have been constructed by self-assembling of chitosan as polycation and poly-gamma glutamic acids (PGA) as polyanion. The nanoparticles are capable of Gd-ion uptake forming a particle with suitable molecular relaxivity. Folic acid is linked to the nanoparticles to produce bioconjugates that can be used for targeted in vitro delivery to a human cancer cell line.

WO06042146 relates to conjugates comprising a nanocarrier, a therapeutic agent or imaging agent and a targeting agent. Disclosed are conjugated comprising a nanocarrier, a therapeutic agent or imaging agent, and a targeting agent, wherein the nanocarrier comprises a nanoparticle, an organic polymer, or both. Compositions comprising such conjugates and methods for using the conjugates to deliver therapeutic and/or imaging agents to cells are also disclosed. The conjugate is a compound having the following formula: A-X-Y wherein A represents the chemotherapeutic agent or imaging agent; X represents the nanoparticle, organic polymer or both, wherein the organic polymer has an average molecular weight of at least about 1,000 daltons; and Y represents the targeting agent.

WO0016811 relates to an MRI contrast agent wherein imaging capability is expressed only within the target abnormal cells, such as tumor, and imaging is not conducted at the site where imaging is not necessary, thereby the detection sensitivity of the abnormal cells such as tumor is improved. Disclosed is an MRI contrast agent, which comprises a complex of a polyanionic gadolinium (Gd) type contrast agent and a cationic polymer, or a complex of a polycationic Gd type contrast agent and an anionic polymer, both complexes being capable of forming a polyion complex, and which expresses an MRI capability at a neutral pH in the presence of a polymer electrolyte.

The state of the art so far failed to provide for the improved compositions according to the present invention.

### SUMMARY OF THE INVENTION

The present invention is directed to novel, targeting dual-modality PET/MRI or PET/CT tumorspecific contrast agents.

In some embodiments, the fusion nanoparticulate composition comprises (i) at least two polyelectrolyte biopolymers, (ii) targeting molecules conjugated to a polyanion biopolymer, (iii) a complexing agent conjugated to a polycation biopolymer, (iv) an MR or CT active ligand complexed to the nanoparticles, and (v) a radionuclide complexed to the nanoparticles.

The MR active ligands can be gadolinium ions as T1 MR active ions, superparamagnetic iron oxide nanoparticles (SPION) as T2 MR active ligands, or gold nanoparticles as CT contrast ligand.

Gadolinium ions are complexed to the nanoparticles via complexing agents conjugated to a polycation biopolymer. SPION and gold nanoparticles are formed in presence of a polyelectrolyte biopolymer to produce complexed ligands.

In a preferred embodiment, the polycation biopolymer is preferably chitosan; and the polyanion biopolymer is preferably poly-gamma-glutamic acid.

In a further embodiment, the chitosan of the nanoparticles ranges in molecular weight from about 20 kDa to 600 kDa, and the poly-gamma-glutamic acid of the nanoparticles ranges in molecular weight from about 50 kDa to 2500, preferably 1500 kDa. In a preferred embodiment, the degree of deacetylation of chitosan ranges between 40% and 99%.

Targeting moieties are conjugated to polyanion to realize a targeted delivery of imaging agents.

The targeting agent is preferably folic acid, LHRH, RGD.

The self-assembled nanosystems contain complexing agents. The polycation modified by the complexing agent allows the chelation of gallium for PET imaging and allows the chelation of gadolinium for MR imaging. Preferable complexing agents include, but are not limited to: diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA).

In a further embodiment, the nanoparticles have a mean particle size between about 30 and 500 nm, preferably between about 50 and 400 nm, and most preferably between 70 and 250 nm.

Accordingly, the invention concerns a targeting PET/MRI or PET/CT tumorspecific nanoparticulate contrast composition comprising (i) at least two, preferably water-soluble, biocompatible and biodegradable nanoparticle polyelectrolyte biopolymers; (ii) a targeting molecule conjugated a polyanion biopolymer; (iii) a complexing agent conjugated to a polycation biopolymer, (iv) an MR or CT active ligand complexed to the nanoparticles, and (v) a radionuclide, preferably gallium complexed to the nanoparticles.

Furthermore, the invention relates to a process for the preparation of a targeting contrast composition according to the invention, comprising the steps of

a) contacting of a solution comprising the polyanion, the targeting agent and the MR or CT active ligand; with the conjugate of the polycation and the complexing agent; and

b) labeling of the conjugate of the polycation and the complexing agent or the self-assembled nanoparticles.

Still further, the invention relates to the use of the contrast composition according to the invention as fusion PET/MR or PET/CT imaging agents in diagniosis, preferably cancer diagnosis.

The present invention provides fusion PET/MR or PET/CT imaging agents that are compositions comprising radioactively labeled MR or CT active nanoparticles. The compositions of the invention target tumor cells, selectively internalize and accumulate in them in consequence of the presence of targeting ligands, therefore are suitable for early tumor diagnosis.

### BRIEF DESCRIPTION OF THE SEVERAL VIEWS OF THE DRAWINGS

FIG. 1 shows the size and size distribution of self-assembled nanoparticles.

FIGS. 2A and 2B show the T₁-weighted MRI images of non Gd conjugated self-assembled nanoparticles SI=301 (FIG. 2A) and Gd conjugated self-assembled nanoparticles SI=1486 (FIG. 2B). The Gd-NPs show a significant contrast enhancement, which is exhibited in the high signal intensity.

FIGS. 3A and 3B show the chromatogram of normal generator-eluted ⁶⁸Ga solution (FIG. 3A) and ⁶⁸Ga-NPs (FIG. 3B). Free, unbound Ga-68 was migrated with the solvent to the front line (Rf=1), while the labeled nanoparticle compound was located at the origin (Rf=0). Integrating measured peaks showed the proper ratios of labeled and non-labeled components.

FIG. 4 shows uptake percent of total activity of self-assembled nanoparticles radiolabeled with ⁶⁸Ga radionuclide on KB cells.

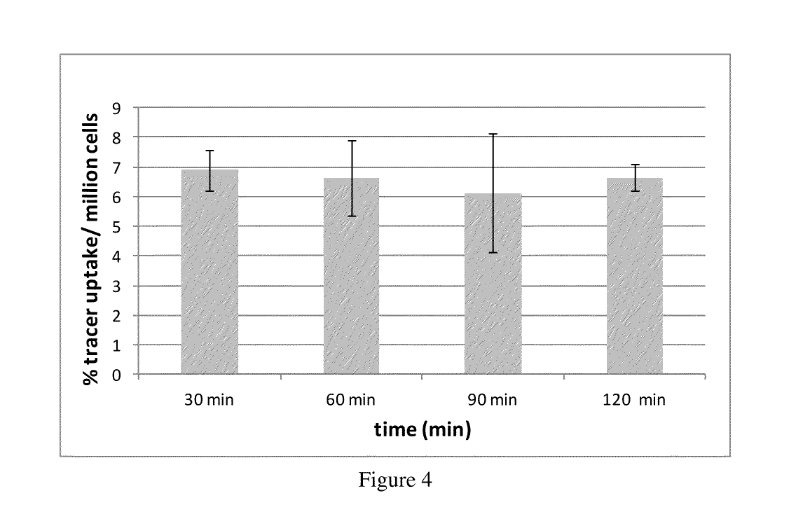

### DETAILED DESCRIPTION OF THE INVENTION

The present invention provides novel, targeting, dual-modality PET/MRI or PET/CT tumorspecific contrast agent and method for forming them for targeted delivery. Self-assembled particles are provided as nanocarriers, labeled with targeting moieties, containing complexone ligands conjugated to a polycation biopolymer, MR or CT active ligand complexed to the nanoparticles, and radionuclide complexed to the nanoparticles. Methods for making these targeting dual-modality contrast agents are also provided.

Nanoparticles, as Contrast Agent Compositions

The present invention is directed to biocompatible, biodegradable, polymeric nanoparticles, as dual-modality tumorspecific contrast agent, formed by self-assembly via the ion-ion interaction of oppositely charged functional groups of polyelectrolyte biopolymers, as nanocarriers for PET and MRI or CT active ligands.

In a preferred embodiment, the biopolymers are water-soluble, biocompatible, biodegradable polyelectrolyte biopolymers. One of the polyelectrolyte biopolymers is a polycation, a positively charged polymer, which is preferably chitosan or any of its derivatives. The other of the polyelectrolyte biopolymers is a polyanion, a negatively charged biopolymer. The polyanion is preferably selected from a group consisting of polyacrylic acid (PAA), poly-gamma-glutamic acid (PGA), hyaluronic acid (HA), and alginic acid (ALG).

In a preferred embodiment, the polycation of the nanoparticles ranges in molecular weight from about 20 kDa to 600 kDa, and the polyanion of the nanoparticles ranges in molecular weight from about 50 kDa to 2500, preferably 1500 kDa.

In a preferred embodiment, the degree of deacetylation of chitosan ranges between 40% and 99%.

The nanoparticles contain targeting moieties necessary for targeted delivery of nanosystems.

The targeting agent is coupled covalently to one of the biopolymers using a carbodiimide technique in aqueous media. The water soluble carbodiimide, as coupling agent forms amide bonds between the carboxyl and amino functional groups, therefore the targeting ligand could be covalently bound to one of the polyelectrolyte biopolymers.

In the present invention, the preferred targeting agent is selected from folic acid, lutenizing hormone-releasing hormone (LHRH), and an Arg-Gly-Asp (RGD)-containing homodetic cyclic pentapeptide such as cyclo(-RGDf(NMe)V) and the like.

In a preferred embodiment, the most preferred targeting agent is folic acid, which facilitates the folate mediated uptake of nanoparticles, as tumor specific contrast agents. The nanoparticles of the present invention are preferably targeted to tumor and cancer cells, which overexpress folate receptors on their surface. Due to the binding activity of folic acid ligands, the nanoparticles selectively link to the folate receptors held on the surface of targeted tumor cells, internalize and accumulate in the tumor cells.

Folic acid is coupled covalently to the polyanion biopolymer using a carbodiimide technique. The folic acid due to its carboxyl and amino groups can be coupled to the polyanion biopolymer directly or via a PEG-amine spacer.

In a preferred embodiment, the self-assembled nanoparticles are comprised of a polyanion biopolymer, a polycation biopolymer, a targeting agent covalently attached to one of the biopolymers and at least one complexing agent covalently coupled to the polycation.

The complexing agent is coupled covalently to the polycation biopolymer. Water-soluble carbodiimide, as coupling agent is used to make stable amide bonds between the carboxyl and amino functional groups in aqueous media. Using reactive derivatives of complexing agents (e.g. succinimide, thiocyanete), the polycation-complexone conjugate can be directly formed in one-step process without any coupling agents. The nanoparticles can make stable complex with the radionuclide metal ions and for PET/MRI T1 modality, paramagnetic ions through these complexone ligans.

In a preferred embodiment, the complexing agents are preferably diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), 1,4,7-triazacyclononane-N,N′,N″-triacetic acid (NOTA) or their reactive derivatives. More preferably, the complexing agents are DOTA, DTPA, EDTA and NOTA, most preferably DTPA for paramagnetic ligand and NOTA for radionuclide metal ions.

The targeted, dual-modality self-assembled nanoparticles described herein are radiolabeled with radionuclide metal ion, which is preferably ⁶⁸Ga to realize the PET modality.

In a preferred embodiment, the radionuclide metal ions are homogeneously distributed throughout the self-assembled nanoparticle. The radionuclide metal ions can make stable complex with the free complexing agents attached to the polycation biopolymer, therefore they could be performed homogeneously dispersed.

To achieve the dual-modality PET/MR tumorspecific contrast agents, T1 or T2 ligands are conjugated to the nanocarriers, and thereafter radiolabelling with radionuclide gallium is carried out.

For T1 MRI modality, paramagnetic ions are complexed to the nanocarriers. The paramagnetic ions are preferably lanthanide or transition metal ions, more preferably gadolinium-, manganese-, chromium-ions, most preferably gadolinium ions, useful as MRI contrast agent.

The paramagnetic ions are homogeneously distributed throughout the self-assembled nanoparticle.

The paramagnetic ions can make stable complex with the complexone ligands attached to the polycation biopolymer; therefore they could be performed homogeneously dispersed.

For T2 modality, superparamagnetic ligand, preferably superparamagnetic iron oxide nanoparticles are conjugated to a polyelectrolyte biopolymer, and they are preferably homogenously dispersed. The superparamagnetic iron oxide nanoparticles (SPION) are synthesized in situ in the presence of the polyanion, and then the self-assembling with the polycation is performed.

The size of the dried SPIONs ranges between 1 and 15 nm, preferably 3 and 5 nm.

To achieve the dual-modality PET/CT tumorspecific contrast agents, gold nanoparticles are conjugated to the nanocarriers, and thereafter radiolabelling with radionuclide gallium is carried out.

The gold nanoparticles are synthesized in situ in the presence of the polyanion, and then the self-assembling with the polycation is performed.

In a preferred embodiment, the nanoparticles described herein have a hydrodynamic diameter between about 30 and 500 nm, preferably between about 50 and 400 nm, and the most preferred range of the hydrodynamic size of nanoparticles is between 70 and 250 nm.

Methods of Making Nanoparticles, as Dual-Modality Contrast Agent Compositions

The present invention is directed to novel, biocompatible, biodegradable, targeting nanoparticles as dual-modality PET/MRI or PET/CT contrast agents. The nanoparticle compositions described herein are prepared by the self-assembly of oppositely charged polyelectrolytes via ion-ion interaction between their functional groups. The targeting ligands are conjugated covalently to one of the polyelectrolyte biopolymers and complexing agents covalently coupled to the polycation biopolymer.

These nanoparticles can contain paramagnetic ligand as MRI T1, superparamagnetic ligands as MRI T2 agents or gold nanoparticles as CT active ligands. These targeted nanoparticles are radioactively labeled with ⁶⁸Ga radionuclide to produce dual-modality fusion contrast agents.

In a preferred embodiment, the targeting ligand is attached to one of the biopolymers covalently. The targeting agent is preferably folic acid, LHRH, RGD, the most preferably folic acid.

The folic acid is coupled covalently to the polyanion biopolymer using a carbodiimide technique. The folic acid due to its carboxyl and amino groups can be coupled to the polyanion biopolymer directly or via a PEG-amine spacer.

The polyanions via their reactive carboxyl functional groups can form stable amide bond with the amino functional groups of folic acid or the folic acid-PEG amino spacer using carbodiimide technique. Folated biopolymer meaning folated polyanion can be used for the formation of nanoparticles, as targeted dual-modality contrast agent.

In a preferred embodiment, the polycation-complexone polyelectrolyte derivatives are used for the formation of self-assembled nanoparticles. These derivatives of the polycation are produced by coupling complexing agent to it covalently. Water soluble carbodiimide is used as coupling agent to form stable amide linkage between the amino groups of polycation and carboxyl groups of complexing agent. Using reactive derivatives of complexing agents (e.g. succinimide, thiocyanete), the polycation-complexone conjugate can be directly formed in one-step process without any coupling agents. In the present invention several complexing agent having reactive carboxyl groups are used to make stable complex with metal ions and therefore afford possibility to use these systems as imaging agent.

For the formation of conjugation, the concentration of the biopolymer ranges between about 0.05 mg/ml and 5 mg/ml, preferably 0.1 mg/ml and 2 mg/ml, and the most preferably 0.3 mg/ml and 1 mg/ml.

The overall degree of substitution of the compexing agent in polycation-complexone conjugate is generally in the range of about 1-50%, preferably in the range of about 5-30%, and most preferably in the range of about 10-20%.

Two types of polycation-complexone conjugate can be used for the formation of nanoparticles: (i) a polycation-complexone conjugate, when the complexing agent specific to the radionuclide is covalently attached to the polycation; and (ii) a polycation-complexone conjugate, when two different complexing agents are covalently coupled to the polycation biopolymer, one of them is specific to the paramagnetic ligand and the other is to the radionuclide.

In a preferred embodiment, nanoparticulate compositions, as targeted, dual-modality PET/MRI T1 contrast agents are provided. The T1 MR active agent is a paramagnetic ligand, which is preferably a lanthanide or transition metal ion, more preferably a gadolinium-, a manganese-, a chromium-ion, most preferably a gadolinium ion, useful for MRI. The preferred paramagnetic ions can make stable complex with the targeting, self-assembled nanoparticles due to the complexing agents covalently conjugated to polycation.

The gadolinium-chloride solution was used as simple aqueous solution without any pH adjusting. In a preferred embodiment, concentration of gadolinium ion ranges between about 0.2 mg/ml and 1 mg/ml, most preferably between 0.4 mg/ml and 0.5 mg/ml. The molar ratio of said gadolinium ions and complexone conjugated to the polycation ranges preferably between 1:10 and 1:1, more preferably 1:5 and 1:1, and most preferably 1:1.

In a preferred embodiment, nanoparticulate compositions, as targeted, dual-modality PET/MRI T2 contrast agents are provided. The T2 MR active agent is a superparamagnetic ligand, preferably iron-oxide ligand, which is preferably nanoparticulate iron-oxide (SPION), which is complexed to a polyelectrolyte biopolymer, and preferably homogenously dispersed.

The superparamagnetic iron oxide nanoparticles are produced in situ in presence of polyanion or targeted polyanion biopolymers, therefore superparamagnetic iron oxide particles are coated by a polyelectrolyte biopolymer.

The SPION synthesis can be performed using several types of Fe(III) and Fe(II) ions, such as pl. FeCl₃xnH₂O (hydrate), Fe₂(SO₄)₃, Fe(NO₃)₃, Fe(III)-phosphate, FeCl₂xnH₂O, FeSO₄xnH₂O (hydrate), Fe(II)-fumarate, or Fe(II)-oxalate.

Preferably, the concentration of polyanion is between 0.01-2.0 mg/ml, the ratio of Fe(III) and Fe(II) ions ranges between 5:1 and 1:5. The reaction takes place at elevated temperature ranging between 45 and 90° C. under N₂ atmosphere.

In a preferred embodiment, nanoparticulate compositions, as targeted, dual-modality PET/CT contrast agents are provided. The CT active ligands are gold nanoparticles with size range of 2-15 nm, preferably 5-12 nm. The gold nanoparticles are produced in situ in the presence of a polyanion or a targeted polyanion biopolymer, therefore the gold nanoparticles are homogenously dispersed and coated by the polyelectrolyte biopolymer.

Preferably, the concentration of polyanion is between 0.01-3.0 mg/ml, the molar ratio of AuCl₃ and polyanion monomers ranges between 2:1 and 5:1. Synthesis of gold nanoparticles in situ in presence of polyanion may be performed using sodium borohydride as reducing agent and optionally sodium citrate dehydrate as stabilizing agent. The molar ratio of gold chloride, sodium borohydride and optionally sodium citrate dehydrate is 1:1:1.

For production of dual modality contrast agents, the T1 MR, T2 MR or CT active ligand bearing nanoparticles are radioactively labeled with a PET active radionuclide ligand, which is preferably ⁶⁸Ga ion. The preferred radioactive metal ions can make stable complex with the targeting, self-assembled nanoparticles due to the complexing agents, which are covalently conjugated to polycation.

In the last step, targeted, self-assembled nanoparticles are radiolabeled with ⁶⁸Ga to produce dual modality radiodiagnostic imaging agents. The radiolabeling takes place in HEPES solution. For labeling, a ⁶⁸Ge/⁶⁸Ga generator is eluted with 1 M ultra pure HCl. The second fraction is buffered with 800 μl HEPES buffer solution and 25% ultra pure NaOH to ensure a pH of 6.4-6.6. Thereafter an aqueous solution of nanoparticle is added to the solvent. The incubation temperature for radiolabeling is room temperature, the incubation time for radiolabeling ranges preferably between 2 min and 60 min, more preferably 5 min and 30 min, and the most preferably 15 min. The raw product is purified using mPES MicroKros Filter Module (10 kD, Spectrumlabs) and osmolarity is adjusted to 280+−10 mOsm/L with 5% glucose solution.

The nanocarrier formation of the present invention can be obtained in several steps. For production of PET/MR T1 dual-modality contrast agent, solution targeted polyanion and polycation-complexone are mixed to form stable, self-assembled nanoparticles, and after that aqueous solution of paramagnetic ions is added to these nanoparticles to make stable paramagnetic nanoparticulate contrast agent. Thereafter these paramagnetic nanoparticles are radioactively labeled with ⁶⁸Ga PET active radionuclide metal ions to produce the fusion contrast agent.

For the production of PET/MR T2 dual-modality contrast agent, solution of targeted, a SPON-loaded polyanion and a polycation-complexone are mixed to form stable, superparamagnetic self-assembled nanoparticles. Then these superparamagnetic nanoparticles are radioactively labeled with ⁶⁸Ga PET active radionuclide metal ions to produce the fusion contrast agent.

For the production of a PET/CT dual-modality contrast agent, a solution of the targeted, gold nanoparticles-loaded polyanion and the polycation-complexone are mixed to form stable, superparamagnetic self-assembled nanoparticles. Then these CT active nanoparticles are radioactively labeled with ⁶⁸Ga PET active radionuclide metal ions to produce the fusion contrast agent.

The nanoparticle compositions of present invention are prepared by mixing of the aqueous solution of biopolymers at given ratios and order of addition. The polyelectrolytes have statistical distribution inside the nanoparticles to produce globular shape of the nanosystems.

The size of nanoparticles can be controlled by several reaction conditions, such as the concentration of biopolymers, the ratio of biopolymers, and the order of addition. The charge ratio of biopolymers depends on the pH of the environment. In preferred embodiment, the pH of the polycation or its derivatives varies between 3.5 and 6.0, and the pH of the aqueous solution of polyanion or its derivatives ranges between 7.5 and 9.5.

Biopolymers with high charge density form stable nanoparticles due to these given pH values. The surface charge of nanoparticles could be influenced by several reaction parameters, such as ratio of biopolymers, ratio of residual functional groups of biopolymers, pH of the biopolymers and the environment, etc. The electrophoretic mobility values of nanoparticles, showing their surface charge, could be positive or negative, preferably negative, depending on the reaction conditions described above.

In a preferred embodiment, the concentration of biopolymers ranges between about 0.005 mg/ml and 2 mg/ml, preferably between 0.2 mg/ml and 1 mg/ml, most preferably 0.3 mg/ml and 0.5 mg/ml. The concentration ratio of biopolymers mixed is about 2:1 to 1:2, most preferably about 1:1. The biopolymers are mixed in a weight ratio of 6:1 to 1:6, most preferably 3:1 to 1:3.

Methods of Using Nanocarrier Compositions

The radiolabeled, targeting dual-modality nanoparticle compositions are useful for targeted delivery of radionuclide metal ions MR or CT active ligands coupled or complexed to the nanoparticles. The present invention is directed to methods of using the above-described nanoparticles, as targeted, dual-modality PET/MR or PET/CT contrast agents.

In a preferred embodiment, the nanoparticles as nanocarriers deliver the imaging agents to the targeted tumor cells in vitro, therefore can be used as targeted, dual-modality PET/MR or PET/CT contrast agents. The radiolabeled nanoparticles internalize and accumulate in the targeted tumor cells, which overexpress folate receptors, to facilitate the early tumor diagnosis. The side effect of these contrast agents is minimal, because of the receptor mediated uptake of nanoparticles.

In a preferred embodiment, the radioactively labeled, targeted dual-modality imaging agents are stable at pH 7.4, they may be injected intravenously. Based on the blood circulation, the nanoparticles could be transported to the area of interest.

The osmolarity of nanosystems was adjusted using formulating agents. The formulating agent was selected from the group of glucose, physiological salt solution, phosphate buffered saline (PBS), sodium hydrogen carbonate and other infusion base solutions.

The ability of the radiopharmaceutical, dual-modaity nanoparticles to be internalized was studied in cultured cancer cells, which overexpresses folate receptors using confocal microscopy and flow cytometry.

Specific localization, accumulation and biodistribution of these radioactively labeled targeted nanoparticles were investigated in vivo using tumor induced animal. Targeted, radiolabeled nanoparticles specifically internalize into the tumor cells overexpressing folate receptors on their surface. The specific localization was examined by PET/MR and PET/CT methods, and the biodistribution was estimated by quantitative ROI analysis.

### EXAMPLES

### Example 1

### Preparation of Folated Poly-Gamma-Glutamic Acid (γ-PGA)

Folic acid was conjugated via the amino groups to γ-PGA using carbodiimide technique: γ-PGA (m=300 mg) was dissolved in water (V=300 ml) to produce aqueous solution at a concentration of 1 mg/ml. The pH of the polymer solution was adjusted to 6.0. After addition of 1-hydroxybenzotriazole hydrate (m=94 mg), the reaction mixture was sonicated for 5 min. The reaction mixture was cooled to 4° C. and cold water-soluble 1-[3-(dimethylamino)propyl]-3-ethylcarbodiimide hydrochloride (EDC) (m=445 mg in V=15 ml water) was added dropwise to the γ-PGA aqueous solution. The reaction mixture was stirred at 4° C. for 10 min, then folic acid (FA) solution (m=69 mg in V=15 ml water) and triethylamine (V=324 μl) were added dropwise to the reaction mixture. The reaction mixture was stirred for 24 h. The folated poly-γ-glutamic acid (γ-PGA-FA) was purified using mPES MicroKros Filter Module (10 kD).

### Example 2

### Preparation of Folated Poly-Gamma-Glutamic Acid

Synthesis of folated PGA was performed in a two steps process. First PEG amine was coupled to FA based on a well-known reaction described in the literature. [JACS, 130 (2008) 11467] After that FA-PEG amine was conjugated via the amino groups to PGA using the carbodiimide technique: γ-PGA (m=300 mg) was dissolved in water (V=300 ml) to produce aqueous solution at a concentration of 1 mg/ml. The pH of the polymer solution was adjusted to 6.0. After addition of 1-hydroxybenzotriazole hydrate (m=94 mg), the reaction mixture was sonicated for 5 min The reaction mixture was cooled to 4° C. and cold water-soluble 1-[3-(dimethylamino)propyl]-3-ethylcarbodiimide hydrochloride (EDC) (m=445 mg in V=15 ml water) was added dropwise to the γ-PGA aqueous solution. The reaction mixture was stirred at 4° C. for 10 min, then folic acid-PEG-amine solution (m=100 mg in V=15 ml water) and triethylamine (V=324 μl) were added dropwise to the reaction mixture. The reaction mixture was stirred for 24 h. The folated poly-y-glutamic acid (γ-PGA-PEG-FA) was purified using mPES MicroKros Filter Module (10 kD).

### Example 3

### Preparation of Folated Poly-Gamma-Glutamic acid coated iron oxide (PFS)

The pH of the folated PGA solution (c=0.3 mg/ml, V=30 ml) was adjusted to 2.8. After the dropwise addition of FeCl₃x6H₂O solution (c=0.5 mg/ml, V=13.9 ml), the pH of the reaction mixture was raised to 8.5 and after that it was reduced to 6.0. The reaction mixture was stirred for 30 min under N₂ atmosphere, and FeCl₂x4H₂O (m32 8.9 mg) was added to the reaction mixture. Reaction temperature was raised to 80° C. and the pH was raised by addition of ammonium solution (V=3 ml, c=12.5 m/m%). Reaction time is 15 min.

### Example 4

### Preparation of Folated Poly-Gamma-Glutamic Acid Coated Gold Nanoparticles

Folated PGA was dissolved in distilled water (V=10 ml) to produce a solution with a concentration of c=0.5 mg/ml. After the dropwise addition of solution of gold (III) chloride hydrate (V=500 μl, c=1.7 mg/ml), solution of sodium citrate tribasic dihydrate (V=75 μl, c=10 mg/ml) was added dropwise to the reaction mixture. Then solution of sodium borohydride (V=40 μl, c=1 mg/ml) was added to the reaction. The reaction mixture was stirred at room temperature for 4 h, after that it was purified by dialysis.

### Example 5

### Preparation of Chitosan-EDTA Conjugate

Chitosan (m=15 mg) was solubilized in water (V=15 ml); its dissolution was facilitated by dropwise addition of 0.1 M HCl solution. After the dissolution, the pH of chitosan solution was adjusted to 5.0. After the dropwise addition of EDTA aqueous solution (m=11 mg, V=2 ml), the reaction mixture was stirred at room temperature for 30 min, and at 4° C. for 15 min after that, CDI (m=8 mg, V=2 ml distilled water) was added droppwise to the reaction mixture and stirred 4° C. for 4 h, then at room temperature for 20 h. The chitosan-EDTA conjugate (CH-EDTA) was purified by dialysis.

### Example 6

### Preparation of Chitosan-EDTA-NOTA Conjugate

The pH of the chitosan-EDTA solution (c=0.5 mg/ml, V=10 ml) was adjusted to 6.1. NODA-GA-NHS ester 10 mg was dissolved in 1 ml DMSO. The NODA-GA-NHS solution (c=10 mg/ml, V=230 μl) was added dropwise to chitosan-EDTA solution and the reaction mixture was stirred at room temperature for 24 h. The chitosan-EDTA-NOTA conjugate (CH-EDTA-NOTA) was purified by dialysis.

### Example 7

### Preparation of Self-Assembled MRI (T1) Nanoparticles

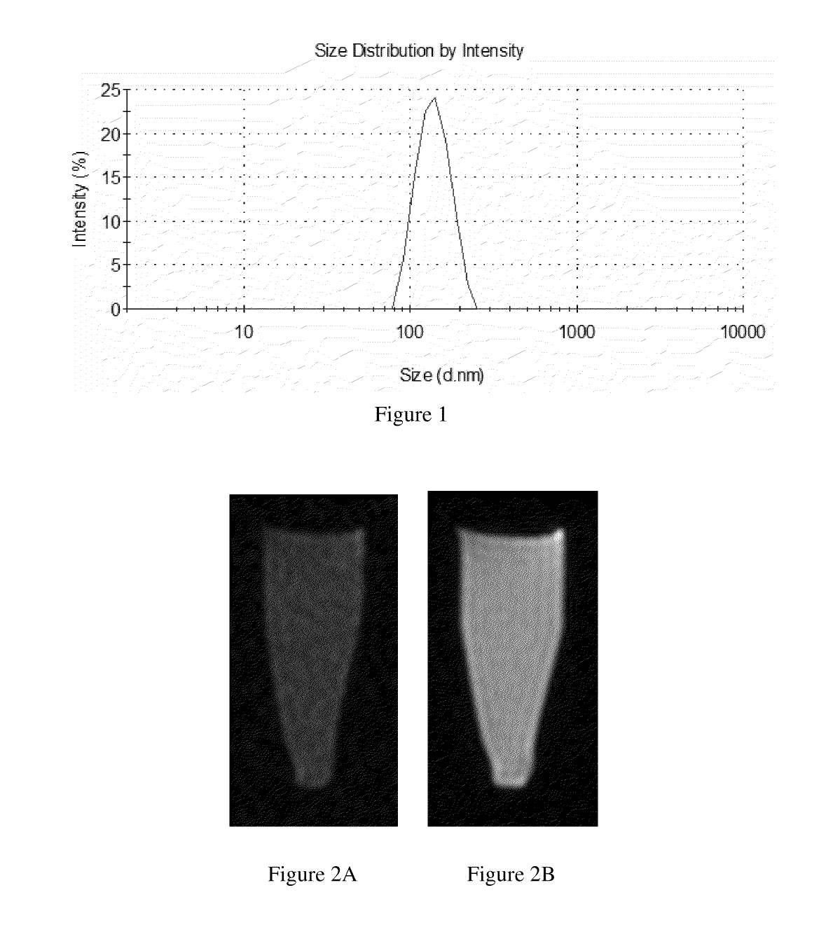

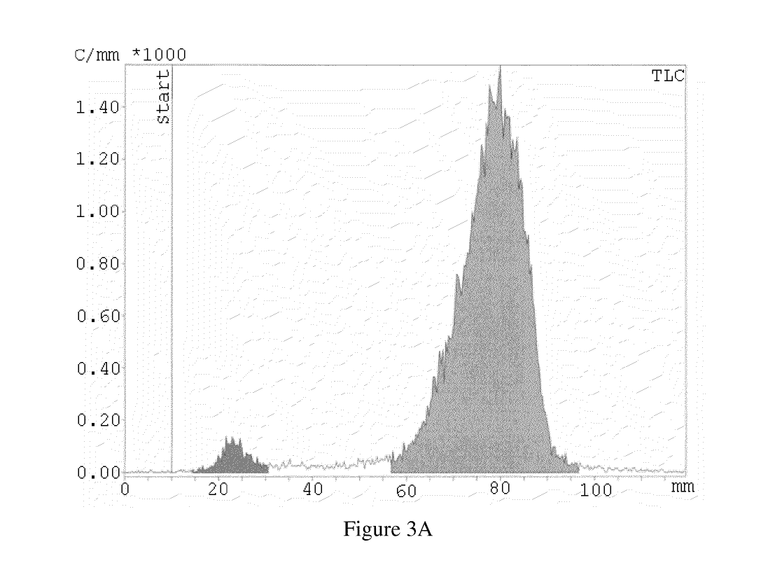

Stable self-assembled nanoparticles were developed via an ionotropic gelation process between the folated poly-γ-glutamic acid (γ-PGA-FA) and chitosan-EDTA-NOTA conjugate. Briefly, CH-EDTA-NOTA solution (c=0.3 mg/ml, V=1 ml, pH=4.0) was added into γ-PGA-FA solution (c=0.3 mg/ml, V=1 ml, pH=9.0) under continuous stirring. An opaque aqueous colloidal system was gained, which remained stable at room temperature for several weeks at physiological pH. (FIG. 1) After radioactive labeling, Gd-ions were added to the nanosystem to produce fusion PET/MR T1 contrast agent. (FIG. 2)

### Example 8

### Preparation of Self-Assembled MRI (T2) Active Nanoparticles

CH-EDTA-NOTA solution (c=0.3 mg/ml, V=1 ml, pH=4.0) was added into folated poly-gamma-glutamic acid coated iron oxide (PFS) solution (c=0.3 mg/ml, V=2 ml, pH=9.0) under continuous stirring.

### Example 9

### Preparation of Self-Assembled CT Active Nanoparticles

CH-EDTA-NOTA solution (c=0.2 mg/ml, V=1 ml, pH=4.0) was added into folated poly-gamma-glutamic acid coated gold nanoparticle solution (c=0.2 mg/ml, V=3 ml, pH=9.0) under continuous stirring.

### Example 10

### Labeling Method of Self-Assembled Nanoparticles

A ⁶⁸Ge/⁶⁸Ga generator was eluted with 1.5 ml fractions of 1 M ultra pure HCl. The second 1250 μl fraction (280+−20 MBq) was buffered with 800 μl HEPES buffer solution (7.2 g HEPES was dissolved in 6 ml ultra-pure water) and 155 μl 25% ultra pure NaOH to ensure a pH of 6.4-6.6. Thereafter an aqueous solution of NOTA-Nanoparticle compound (V=245 μl c=0.3 mg/ml) was added to the solvent. The mixture was incubated at room temperature for 15 min The raw product was purified using mPES MicroKros Filter Module (10 kD, Spectrumlabs) and Osmolarity was adjusted to 280+−10 mOsm/L with 5% glucose solution.

### Example 11

### Characterization of ⁶⁸Ga Labeled Self-Assembled Nanoparticles

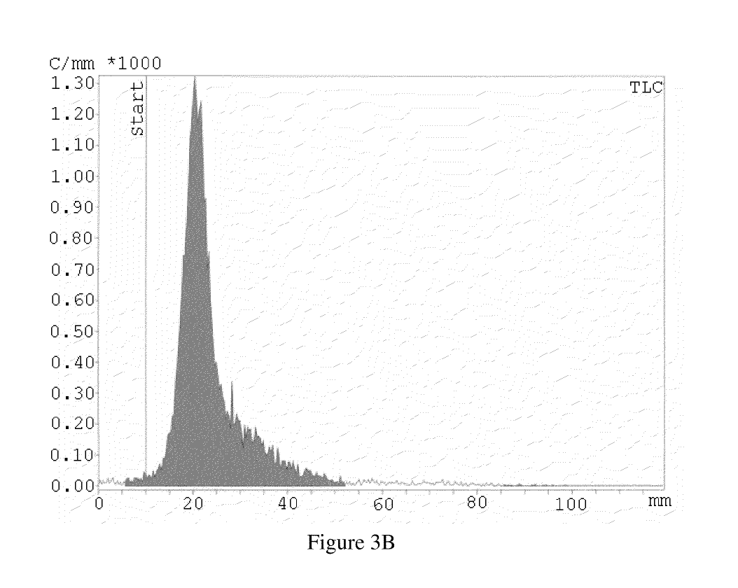

Radiochemical purity was examined by means of thin layer chromatography, using silica gel as the coating substance on a 100 mm glass-fibre sheet (ITLC-SG). Plates were developed in 0.1M Na-citrate. We applied Raytest MiniGita device (Mini Gamma Isotope Thin Layer Analyzer) to determine the distribution of radioactivity in developed ITLC-SG plates. Normal generator-eluted 68Ga solution was used as control. We examined labelling efficiency 30 min after labeling. Radiochemical samples were stored at RT in dark place. The radiolabeled products showed high degree and durable labelling efficiency (above 99%). (FIG. 3)

1. A targeting PET/MRI or PET/CT tumorspecific nanoparticulate contrast composition comprising (i) at least two, preferably water-soluble, biocompatible and biodegradable nanoparticle polyelectrolyte biopolymers; (ii) a targeting molecule conjugated a polyanion biopolymer; (iii) a complexing agent conjugated to a polycation biopolymer, (iv) an MR or CT active ligand complexed to the nanoparticles, and (v) a radionuclide, preferably gallium complexed to the nanoparticles.

2. The targeting contrast composition as claimed in claim 1, wherein the MR or CT active ligand is a paramagnetic ion, preferably a lanthanide or a transition metal ion, more preferably a gadolinium-, a manganese- or a chromium-ion, most preferably a gadolinium ion as T1 MR active ion, a superparamagnetic iron oxide nanoparticle (SPION) as T2 MR active ligand, or a gold nanoparticle as CT contrast ligand.

3. The targeting contrast composition as claimed in claim 1, wherein
a) the gadolinium ions are complexed to the nanoparticles via complexing agents conjugated to a polycation biopolymer; or
b) the SPION or gold nanoparticles are formed in presence of a polyelectrolyte biopolymer to produce complexed ligands.

4. The targeting contrast composition as claimed in claim 1, wherein one of the nanoparticle polyelectrolyte biopolymers is a polycation or a derivative thereof, preferably chitosan, and the other one is a polyanion biopolymer or a derivative thereof, preferably selected from the group consisting of polyacrylic acid (PAA), poly-gamma-glutamic acid (PGA) hyaluronic acid (HA), and alginic acid (ALG), preferably poly-gamma-glutamic acid (PGA), said biopolymers being preferably self-assembled based on the ion-ion interactions between their functional groups.

5. The targeting contrast composition as claimed in claim 1, wherein
a) the polycation, preferably the chitosan, has a molecular weight from about 20 and 600 kDa, and the degree of its deacetylation ranges between 40% and 99%;
b) the polyanion, preferably the poly-gamma-glutamic acid (PGA) has a molecular weight between 50 kDa and 2500, peferably 1500 kDa; and/or
c) the targeting agent is conjugated to the polyanion, and is selected from the group of folic acid, LHRH and an Arg-Gly-Asp (RGD)-containing homodetic cyclic pentapeptide such as cyclo(-RGDf(NMe)V), preferably folic acid.

6. The targeting contrast composition as claimed in claim 1, wherein the complexing agent is selected from the group consisting of diethylenetriaminepentaacetic acid (DTPA), 1,4,7,10-tetracyclododecane-N,-N′,N″,N′″-tetraacetic acid (DOTA), ethylene-diaminetetraacetic acid (EDTA), 1,4,7,10-tetraazacyclododecane-N,N′,N″-triacetic acid (DO3A), 1,2-diaminocyclohexane-N,N,N′,N′-tetraacetic acid (CHTA), ethylene glycol-bis(beta-aminoethyl ether)N,N,N′,N′,-tetraacetic acid (EGTA), 1,4,8,11-tetraazacyclotradecane-N,N′,N″,N′″-tetraacetic acid (TETA), and 1,4,7-triazacyclononane-N,N′,N″-triacetic acid (NOTA).

7. The targeting contrast composition as claimed in claim 1, wherein the nanoparticles have a mean particle size between about 30 and 500 nm, preferably between about 50 and 400 nm, and most preferably between 70 and 250 nm.

8. A process for the preparation of a targeting contrast composition as claimed in claim 1, comprising the steps of
a) contacting of a solution comprising the polyanion, the targeting agent and the MR or CT active ligand with the conjugate of the polycation and the complexing agent; and
b) radiolabeling of the self-assembled nanoparticles.

9. A process for the preparation of a targeting contrast composition as claimed in claim 1, comprising the steps of
a) labeling of conjugate of the polycation and the complexing agent; and
b) contacting of a solution comprising the polyanion, the targeting agent and the MR or CT active ligand with the product from step a).

10. The process as claimed in claim 8, wherein
a) a polycation-complexone conjugate is used, where the complexing agent specific to the radionuclide is covalently attached to the polycation; or
b) a polycation-complexone conjugate is used, where two different complexing agents are covalently coupled to the polycation biopolymer, one of them is specific to the MR active paramagnetic ligand and the other is to the radionuclide.

11. The process as claimed in claim 8, wherein
a) the concentration of the biopolymer ranges between about 0.05 mg/ml and 5 mg/ml, preferably 0.1 mg/ml and 2 mg/ml, and the most preferably 0.3 mg/ml and 1 mg/ml; and/or
b) the overall degree of substitution of complexing agent in the polycation-complexone conjugate is in the range of about 1-50%, preferably in the range of about 5-30%, and most preferably in the range of about 10-20%; and/or
c) the concentration of gadolinium ion used ranges between about 0.2 mg/ml and 1 mg/ml, most preferably between 0.4 mg/ml and 0.5 mg/ml; and/or
d) the molar ratio of the gadolinium ions and the complexone conjugated to the polycation ranges preferably between 1:10 and 1:1, more preferably 1:5 and 1:1, and most preferably 1:1; and/or
e) the gold nanoparticles used are in the size range of 2-15 nm, preferably 5-12 nm;
f) the pH of the polycation or its derivatives varies between 3.5 and 6.0, and the pH of the aqueous solution of the polyanion or its derivatives ranges between 7.5 and 9.5.

12. The process for the preparation of a targeting contrast composition as claimed in claim 8, wherein
a) the concentration of the polyanion is between 0.01-2.0 mg/ml, the ratio of the MR active Fe(III) and Fe(II) ions ranges between 5:1 and 1:5; and/or
b) the reaction takes place at elevated temperature ranging between 45 and 90° C. under N₂ atmosphere.

13. The process as claimed in claim 8, wherein the radiolabeling with ⁶⁸Ga takes place in a buffer solution, comprising the steps as follows:
a) a ⁶⁸Ge/⁶⁸Ga generator is eluted with HCl;
b) the second fraction is buffered with a buffer solution and NaOH to ensure a pH of 3.0-6.8, preferably 6.4-6.6;
c) an aqueous solution of nanoparticle is added to the solvent;
at room temperature to elevated temperature as incubation temperature, for the time period of preferably between 2 min and 60 min, more preferably 5 min and 30 min, and the most preferably 15.

14. The process as claimed in claim 8, wherein the preparation takes place in several steps.

15. A method of diagnosis, said method comprising using the targeting contrast composition as claimed in claim 1 as a fusion PET/MR or PET/CT imaging agent.

16. The method as claimed in claim 15, wherein the targeting contrast composition is used in cancer diagnosis.
